(19) United States
(12) Patent Application Publication
(10) Pub. No.: US 2018/0208622 A1
(43) Pub. Date: Jul. 26, 2018

(54) STABLE CRYSTALLINE FORM OF REGADENOSON

(71) Applicant: SUN PHARMACEUTICAL INDUSTRIES LIMITED, Mumbai (IN)

(72) Inventors: Rajamannar THENNATI, Baroda (IN); Shriprakash Dhar DWIVEDI, Baroda (IN); Arunkumar Gulabsingh YADAV, Baroda (IN); Nischalkumar Vinodbhai PATEL, Baroda (IN); Sunil Rajaram KARANKE, Baroda (IN)

(73) Assignee: SUN PHARMACEUTICAL INDUSTRIES LIMITED, Mumbai (IN)

(21) Appl. No.: 15/758,971

(22) PCT Filed: Sep. 10, 2016
(86) PCT No.: PCT/IN2016/050303

§ 371 Date: Mar. 9, 2018

(30) Foreign Application Priority Data
Sep. 11, 2015 (IN) 3485/MUM/2015

(51) Int. Cl. C07H 19/16

(52) U.S. Cl. CPC C07H 19/16; C07B 2200/13

(57) ABSTRACT

The present invention provides crystalline Form S of regadenoson which is substantially free of residual organic solvent and having an X-ray powder diffraction pattern comprising characteristic peak at 10.3, 10.8, 19.0, 21.6 and 25.5±0.2 degrees 2θ.

### RELATED APPLICATIONS

This application claims the benefit of Indian Patent Application no. 3485/MUM/2015 filed on Sep. 11, 2015 which is hereby incorporated by reference.

### FIELD OF THE INVENTION

The present invention provides stable polymorphic form of regadenoson and process for its preparation.

### BACKGROUND OF THE INVENTION

Regadenoson is an A_{2A} adenosine receptor agonist that is a coronary vasodilator indicated for radionuclide myocardial perfusion imaging (MPI) in patients unable to undergo adequate exercise stress. It is approved by US FDA as Lexiscan® intravenous injection. Regadenoson is chemically described as adenosine, 2-[4-[(methylamino)carbonyl]-1H-pyrazol-1-yl]. Its structural formula is:

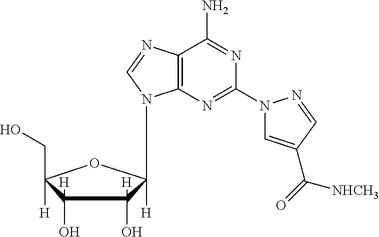

WO2008143667A1 discloses that regadenoson is capable of existing in at least three different crystalline forms (Form A, Form B, Form C) and an amorphous product. It also states that monohydrate Form A is most stable form and polymorph C is a variable hydrate, which, at elevated temperatures, desolvates to an unstable form.

US patent application published as US20140045781 A1 discloses another crystalline form of regadenoson referred as Form D.

WO2014167046A1 discloses crystalline polymorphic forms E, F and G of regadenoson and methods for their preparation.

US patent application US20140323712 A1 discloses another crystalline form of regadenoson which is designated as Form E.

WO2014207758 discloses propylene glycol solvate of regadenoson.

Indian patent application IN 2013CH01831 discloses novel process for the preparation of pure and crystalline form of regadenoson monohydrate.

Indian application IN 2011MU01470 discloses novel polymorphic forms of regadenoson.

### SUMMARY OF THE INVENTION

The present invention provides a stable polymorphic Form S of regadenoson which is substantially free of residual organic solvent and having an X-ray powder diffraction pattern comprising characteristic peak at 10.3, 10.8, 19.0, 21.6 and 25.5±0.2 degrees 2θ.

### DESCRIPTION OF THE DRAWINGS

FIG. 1: X-ray powder diffraction (XRPD) of Form S as obtained in example 1.

FIG. 2: XRPD of Form S after 2 months at 40° C. and 75% relative humidity.

FIG. 3: XRPD of Form S after 6 months at 25° C. and 60% relative humidity.

FIG. 4: Differential scanning calorimetry (DSC) curve of Form S as obtained in example 1.

### DESCRIPTION OF THE INVENTION

In one aspect the present invention provides crystalline Form S of regadenoson which is anhydrous and substantially free of residual organic solvent and having an X-ray powder diffraction pattern comprising characteristic peak at 10.3, 10.8, 19.0, 21.6 and 25.5±0.2 degrees 2θ, wherein the Form S is stable when stored at 25° C. and 60% relative humidity for at least 6 months.

The phrase “Substantially free of residual organic solvent” as referred herein means the content of residual solvent in the crystalline form is less than 0.5%.

In one embodiment, the content of residual organic solvent in crystalline Form S is less than 0.2%.

In another embodiment, the residual organic solvent is selected from ethyl acetate or toluene or a mixture thereof.

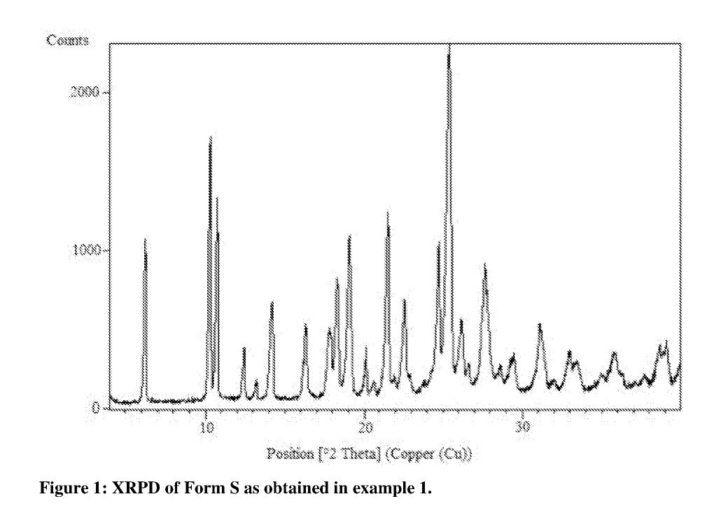

In one embodiment the crystalline Form S is further characterized by X-ray powder diffraction pattern comprising characteristic peak at 6.3, 12.5, 13.2, 14.3, 16.3, 17.8, 18.3, 20.2, 20.7, 22.7, 24.8, 26.2, 26.7, 27.6, 28.6, 29.5, 31.1, 33, 33.5, 35.9, 37.7, 38.6 and 39.1±0.2 degrees 2θ. FIG. 1 provides XRPD of crystalline Form S. Table 1 provides the list of peaks observed in the XRPD pattern along with their intensities.

TABLE 1

| Position (°2 θ) | Relative intensity |
|---|---|
| 6.3 | 46.8 |
| 10.3 | 77.4 |
| 10.8 | 59.8 |
| 12.5 | 15.8 |
| 13.2 | 6.5 |
| 14.3 | 26 |
| 16.3 | 22.8 |
| 17.8 | 21 |
| 18.3 | 35.8 |
| 19 | 52.2 |
| 20.2 | 15.7 |
| 20.7 | 4.1 |
| 21.6 | 58.9 |
| 22.7 | 27.7 |
| 24.8 | 45.8 |
| 25.5 | 100 |
| 26.2 | 20.9 |
| 26.7 | 7.5 |
| 27.6 | 39 |
| 28.6 | 7.1 |
| 29.5 | 10.4 |
| 31.1 | 21.3 |
| 33 | 12 |
| 33.5 | 8.5 |
| 35 | 4.4 |
| 35.9 | 10.2 |
| 37.7 | 2.9 |
| 38.6 | 10.6 |
| 39.1 | 10.4 |

In another embodiment the crystalline Form S has an endotherm at range of about 272° C. to about 279° C. in its Differential Scanning calorimetry (DSC) scan.

The crystal Form S of present invention can be conveniently prepared by subjecting a suspension of regadenoson in a mixture of ethyl acetate and toluene to azeotropic distillation.

In another embodiment regadenoson used as starting material is having water content of more than 0.6%. Azeotropic distillation can be carried out for a time sufficient to completely remove water from the mixture, for example about 5 to 24 hours. The crystalline Form S can be isolated from the suspension thus obtained by filtering the suspension and drying the solid. Drying can be accomplished by the processes known in the art for instance drying under reduced pressure at a temperature of about 30° C. to about 70° C. The ratio of ethyl acetate:toluene in the solvent mixture may alter from 10:90 to 90:10. In another embodiment ratio of ethyl acetate:toluene is 50:50. The crystalline Form S of present invention is substantially free of residual organic solvent. The content of residual solvent in Form S is less than 0.5%, preferably less than 0.2%, more preferably less than 0.1%.

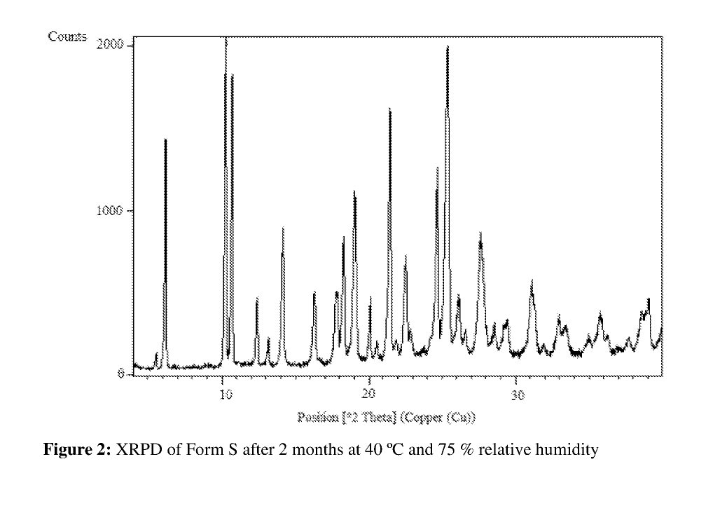

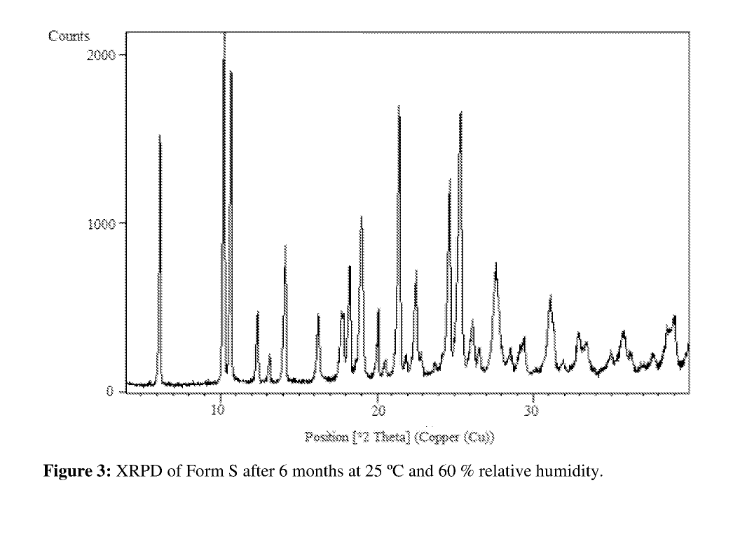

The crystal Form S of present invention was found to be stable during stability studies. For instance, the XRPD of the Form S did not change significantly when the crystal form was subjected to accelerated stability studies at 40° C. and 75% relative humidity for more than 2 months (FIG. 2). There was no significant change in the XRPD of the crystal Form S of the present invention even after subjecting it to stability conditions of 25° C. and 60% relative humidity for more than 6 months (FIG. 3).

The present invention is further illustrated in detail with reference to the following examples. It is desired that the examples be considered in all respect as illustrative and are not intended to limit the scope of the claimed invention.

### EXAMPLES

### Instrument Details:

XRD: X-ray powder diffraction analyses were carried out on a PANalytical Empyrean X-ray powder diffractometer using Cu K alpha radiation. The instrument was equipped with a line focus X-ray tube, and the voltage and amperage were set to 45 kV and 40 mA respectively. The scanning rate was set as 10 second per step and step size is set as 0.01°. The diffractometer was equipped with Pixcel^{1D} solid state detector and rotating sample stage. X-ray diffractometer was used to record diffractogram from 4° to 40° (2-theta).

DSC: Differential Scanning calorimetry (DSC) analysis was performed on a TA Instruments Q2000™. Approximately 2 mg of sample was placed into a tared DSC aluminium pan and sealed hermetically. Typically, the sample was heated under nitrogen at a rate of about 10° C./min from about 35° C. up to a final temperature of about 350° C.

### Example 1: Preparation of Anhydrous Form S of Regadenoson

Regadenoson (10 g) was suspended in a mixture of ethyl acetate:toluene (300 mL, 70:30) under stirring. The suspension was subjected to azeotropic distillation at 80° C. to 85° C. for 12-14 hours. The suspension was cooled to room temperature and the solid was filtered, washed with ethyl acetate (20 mL) and dried under reduced pressure at 65-70° C. for 10-12 hours.

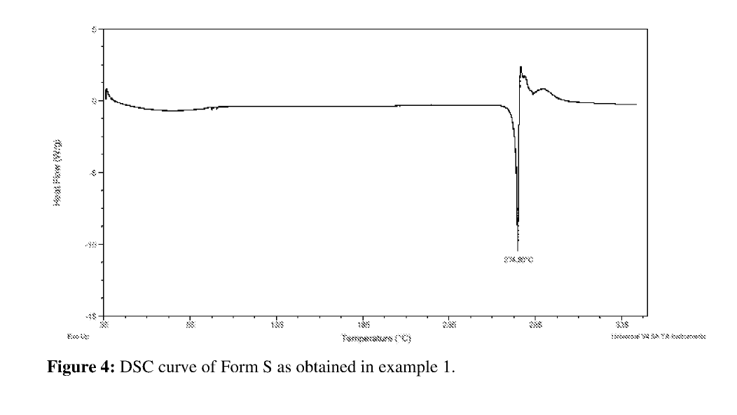

The solid has a XRPD pattern and DSC endotherm substantially same as set forth in FIG. 1 and FIG. 4, respectively. Water content: 0.57% w/w (measured by Karl-Fischer titration method).

1. Crystalline Form S of regadenoson which is anhydrous and substantially free of residual organic solvent and having an X-ray powder diffraction pattern comprising characteristic peak at 10.3, 10.8, 19.0, 21.6 and 25.5±0.2 degrees 2θ, wherein the Form S is stable when stored at 25° C. and 60% relative humidity for at least 6 months.

2. The crystalline Form S as in claim 1 further characterized by X-ray powder diffraction pattern comprising characteristic peak at 6.3, 12.5, 13.2, 14.3, 16.3, 17.8, 18.3, 20.2, 20.7, 22.7, 24.8, 26.2, 26.7, 27.6, 28.6, 29.5, 31.1, 33, 33.5, 35.9, 37.7, 38.6 and 39.1±0.2 degrees 2θ.

3. The crystalline Form S of regadenoson as in claim 1 wherein the content of residual organic solvent is less than 0.5%.

4. The crystalline Form S of claim 1 having a differential scanning calorimetry thermogram comprising an endotherm at about 272° C. to about 279° C.
